(19) United States
(12) Patent Application Publication
(10) Pub. No.: US 2009/0326358 A1
(43) Pub. Date: Dec. 31, 2009

(54) Non-invasive fast-response biodosimeter

(75) Inventor: Henryk Malak, Ellicott City, MD (US)

Correspondence Address: Henryk Malak, 8444 High Ridge Road, Ellicott City, MD, 21043, US

(73) Assignee: American Environmental Systems, Inc

(21) Appl. No.: 11/237,519

(22) Filed: Sep. 29, 2005

Related U.S. Application Data
(63) Continuation-in-part of application No. 10/366,267, filed on Feb. 14, 2003, now Pat. No. 7,081,128.
Application No. 10/366,267 is a continuation-in-part of application No. 10/656,529, filed on Sep. 5, 2003, now Pat. No. 7,049,018.
Application No. 10/656,529 is a continuation-in-part of application No. 10/916,560, filed on Aug. 12, 2004.

(51) Int. Cl. A61B 5/05; A61B 1/00

(52) U.S. Cl. 600/407; 600/101; 250/395; 250/362; 250/363.01

(57) ABSTRACT

Our invention disclosures a biodosimeter and method of measurements a dose of radiation and/or biological-chemical substances absorbed by human or animal body, microbes, plants. The invention describes a multiparametric analysis system to evaluate biochemical and physical patterns related to a range of doses of radiation and/or biological-chemical substances following exposure of human body by radiation and/or biological-chemical substances. In our invention we also propose to use a method of plasmon enhancement by which intrinsic biomolecular targets within human body may increase their fluorescence QY close to unity, especially for the variety of biochemicals. The disclosed robust biodosimeter provides fast-response field applications to normal populations exposed to radiation and/or biological-chemical substances release, and to first-responders evaluating those normal populations.

### CROSS REFERENCE TO RELATED APPLICATIONS

This application is a continuation-in-part of U.S. patent application Ser. No. 10/366,267 filed Feb. 14, 2003 entitled “Joint/Tissue Inflammation Therapy and Monitoring devices”, and a continuation-in-part of U.S. patent application Ser. No. 10/656,529 filed Sep. 8, 2003 entitled “Optochemical Sensing with Multiband Fluorescence Enhanced by Surface Plasmon Resonance”, and a continuation-in-part of U.S. patent application Ser. No. 10/916,560, filed Aug. 12, 2004 entitled “Methods and Devices for Plasmon Enhanced Medical and Cosmetic Procedures”, each of which is incorporated by reference herein in their entirety.

### STATEMENT REGARDING FEDERALLY SPONSORED RESEARCH

There is NO claim for federal support in research or development of this product.

### OTHER REFERENCES

J. D. Eversole, W. K Cary Jr., C. S. Scotto, R. Pierson, M. Spence, and A. J. Campillo, “Optical Detection Capabilities for Biological and Chemical Agent Aerosols” Field Analyt. Chem. Technol. 15, 205 (2001).
I. Gryczynski J. Malicka, Y. Shen, Z. Gryczynski and J. R. Lakowicz, “Multiphoton excitation of fluorescence near metallic particles: enhanced and localized excitation”, J. Phys. Chem. B, 106, 2191 (2002).
S. C. Hill, R. G. Pinnick, P. Nachman, G. Chen, R. K. Chang, M. W. Mayo, and G. L. Fernandez, “Aerosol-fluorescence spectrum analyzer: real-time measurement of emission spectra of airborne biological particles”, Appl. Opt. 34, 7149 (1995).
M. Kasha, “Characterization of electronic transitions in complex molecules”, Discuss. Faraday Soc., 8, 14 (1950).
M. Kerker, “Optics of colloid silver”, J. Colloid Interface Sci. 105, 298 (1985).
K. Kneipp, et al., “Surface-enhanced Raman spectroscopy in single living cells using gold nanoparticles”, Appl. Spectr. 56, 150 (2002).
T. Krupa, “Optical technologies in the fight against bioterrorism” Opt. & Photon. News, 13, 23 (2002), and references herein.
J. R. Lakowicz, Principles of Fluorescence Spectroscopy, Plenum, New York, (1983).
J. R Lakowicz, B. Shen, Z. Gryczynski S. D'Auria, and I. Gryczynski, “Intrinsic fluorescence from DNA can be enhanced by metallic particles”, Biochem. Biophys. Res. Comm. 286, 875 (2001).
R. G. Pinnick, P. Nachman, G. Chen, R. K. Chang, M. W. Mayo, and G. L. Fernandez, “Real-time measurement of fluorescence spectra from single airborne biological particles”, Field Anat. Chem. Technol. 3, 221 (1999), and refs herein.
M. Ratner, and D. Ratner, Nanotechnology, a gentle introduction to the next big idea, Prentice Hall, Upper Saddle River, (2003).
C. Rowe-Tait, Hazzard, J. W., Hoffman, K. E., Cras, J. J., Golden, J. P. and Ligler, F. S Simultaneous detection of six biohazardous agents using a planar waveguide array biosensor” Biosens. Bioelectron. 15, 579 (2000).
S. Terzieva, et al. “Comparison of methods for detection and enumeration of airborne microorganisms collected by liquid impingement” Appl. Environ. Microbiol 62, 2264 (1996).
T. Vo-Dinh, D. L. Stokes, G. D. Griffin, M. Volkan, U. J. Kim, M. I. Simon, “Surface-enhanced Raman scattering (SERS) method and instrumentation for genomics and biomedical analysis”, J. Raman Spectrosc., 30, 785 (1999), and references herein.

### FIELD OF THE INVENTION

This invention is related to biodosimetry devices and method of the use of them in case of uncontrolled radiation and/or biological-chemical substances release.

### BACKGROUND OF THE INVENTION

Determination of exposures of individuals to uncontrolled radiation and/or biological-chemical substances release will likely involve needs to retrospectively determine biological doses of radiation and/or biological-chemical substances received both internally and externally. There is need of development of a biodosimeter sensitive to external acute radiation and/or biological-chemical substances exposures. Biodosimeters serving to determine the dose received by individuals of the normal population exposed to unknown external radiations and/or biological-chemical substances involve development and application of specific sensing devices. The function of those devices is to provide accurate and rapid radiation and/or biological-chemical substances exposure dose assessment so as to minimize the time required to provide mitigation of damage associated with that determined dose. It is important that such a biodosimeter be able to detect non-invasively over an extended time range (minutes to weeks) the dose and distribution of exposure to external radiation and/or biological-chemical substances, and this with a low false-positive rate and a high positive detection rate. In order to minimize the false positive rate and to accurately determine exposure dose within individuals, it is best to non-invasively measure and analyze biological end points that are instantly established upon exposure, and that are unchanged on the order of weeks after exposure. For example, the changeable and transient nature of most gene-expression-based and protein-expression-based biodosimeters are fatal flaws for the needs of radiation dose determination required in normal populations exposed to nuclear release. Rather, our invention is focused on the response of human or animal body to exposure of radiation and/or biological-chemical substances and providing the robust and stable requirements of a useful biodosimeter.

### SUMMARY OF THE INVENTION

Our invention disclosures a biodosimeter and method of measurements a dose of radiation and/or biological-chemical substances absorbed by human or animal body, microbes, and plants. We propose a multiparametric analysis system to evaluate biochemical and physical patterns related to a range of doses of radiation and/or biological-chemical substances following exposure of human body by radiation.

The robust requirement for our biodosimeter is met by direct analysis of intrinsic biochemical and physical patterns in human body. Use of these patterns-based biochemicals is currently limited to distinguishing between biological and inorganic samples, and between proteins, NADH, flavins and chlorophylls/porphyrins. The lack of sensitivity within this long-emergent class of intrinsic biomolecular detection technology has prevented direct detection and identification of different amino acids, proteins and other biomolecules.

To overcome the problem of low sensitivity, i.e., low QY, of intrinsic proteins and biomolecules, in our invention we propose to use a method by which intrinsic biomolecular targets within human body may increase their fluorescence QY close to unity. We propose to use a multiband fluorescence sensing nanotechnique that is based on: (1) coupling energies of low-excitation state of an intrinsic biomolecule with surface plasmon resonance excitation energy of a metal nanoparticle when the molecule is in close proximity to the metal nanoparticle; (2) creating under such energy coupling new spectroscopic properties of the molecule with emphasis on multithousand-fold fluorescence enhancement. Our promotion of low-excitation states is emphasized as key to enhanced intrinsic fluorescence within the body, especially for the variety of biochemicals.

All the above provides logic and established preamble to the basic premise for our disclosed here biodosimeter, which is that radiation and/or biological-chemical substances affect intrinsic biochemicals in human body in ways that can provide a quantitative dose response over a range of biologically significant doses. The disclosed robust biodosimeter provides fast-response field applications to normal populations exposed to radiation and/or biological-chemical substances release, and to first-responders evaluating those normal populations.

### BRIEF DESCRIPTION OF THE DRAWINGS

FIG. 1. Jablonski electronic diagram showing excitations and emissions from lower and higher excited states.

FIG. 2. An example of a biodosimeter design in which light illuminates a body through an optical lens.

FIG. 3. An example of a biodosimeter design in which light illuminates a body through an optical fiber.

FIG. 4. An example of a biodosimeter design in which light illuminates a body through a microscopic objective.

FIG. 5. An example of a biodosimeter design in which light illuminates an internal organ of a body through an optical fiber and endoscope.

### DETAILED DESCRIPTION OF PREFERRED EMBODIMENTS

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

There is disclosed here a biodosimeter and a method of a multiparametric analysis system evaluating biochemical patterns related to a range of doses of radiation and/or biological-chemical substances following exposure of human body samples by radiation and/or biological-chemical substances.

The robust requirement for the biodosimeter is met by direct analysis of intrinsic fluorescence in human body, i.e., analysis will not depend on applications of fluorescent probes. Most currently deployed optical biosensing methods are based on detection of fluorescence markers incorporated into a range of biomolecules [Krupa, 2002; Terzieva et al, 1996; Rowe-Tait, et al, 2000, Hill at al., 1995; Pinnick et al, 1999; Eversole et al, 2001, Lakowicz, 1983]. These methods require relatively complex sample preparation that can alter the desired results and they suffer from long (20 minutes-2 hours) analysis times. In attempts to avoid these limitations, a separate class of substantially less sensitive biosensors that measures the intrinsic fluorescence of biomolecules or pathogens has been identified [Hill at al., 1995; Pinnic et al, 1999; Eversole et al, 2001] based on the fact that the majority of biological specimens include fluorophores as aromatic amino acids, NADH, flavins and chlorophylls/porphyrins [Pinnick et al, 1999; Eversole et al, 2001, Lakowicz, 1983; Ratner et al., 2003; Vo-Dinh et al., 1999]. Use of these intrinsic fluorescence-based sensors is currently limited to distinguishing between biological and inorganic samples, and between proteins, NADH, flavins and chlorophylls/porphyrins.

The lack of sensitivity within this long-emergent class of intrinsic biomolecular detection technology has prevented direct detection and identification of different amino acids, proteins and other biomolecules. The low-excitation state emission rate for a fluorophore is defined by its natural fluorescence lifetime (as a rule, it does not exceed 10⁹ s⁻¹). This value puts a limit on the rate of nonradiative decay and, consequently, the quantum yield (QY) of fluorescence. It is the low value of QY that prevents fluorescent biomolecules being exploited for their unique signatures.

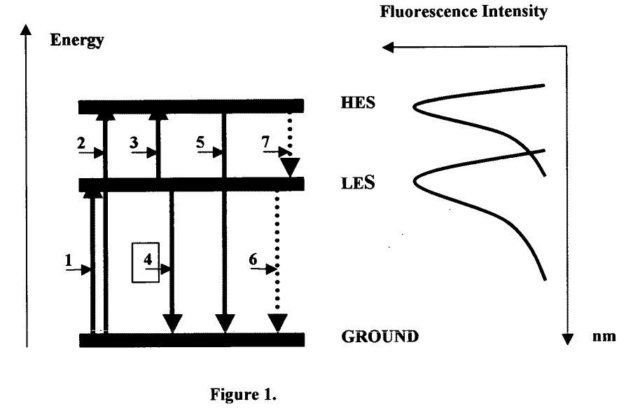

To overcome the problem of low fluorescence, i.e., low QY, of intrinsic proteins and biomolecules, without need for external probes enhancement, we propose to use here a method by which intrinsic biomolecular targets within human body may increase their fluorescence QY close to unity. We propose to use a multiband fluorescence sensing nanotechnique (FIG. 1) that is based on: (1) coupling energies of low-excitation state of an intrinsic biomolecule with surface plasmon resonance excitation energy of a metal nanoparticle when the molecule is in close proximity to the metal nanoparticle; (2) creating under such energy coupling new spectroscopic properties of the molecule with emphasis on multithousand-fold fluorescence enhancement. Our promotion of low-excitation states is emphasized as key to enhanced intrinsic fluorescence within the body, especially for the variety of proteins there, thus complementing nucleic acids already known for 10³ to 10⁴ enhanced intrinsic fluorescence in the presence of metal nanoparticles [Kerker, 1985; Lakowicz et al. 2001; Gryczynski et al., 2002].

All the above provides logic and established preamble to the basic premise for our disclosed here biodosimeter and method, which is that radiation and/or biological-chemical substances affect intrinsic biochemicals in human body in ways that can provide a quantitative dose response over a range of biologically significant doses. The disclosed robust biodosimeter provides fast-response field applications to normal populations exposed to radiation and/or biological-chemical substances release, and to first-responders evaluating those normal populations.

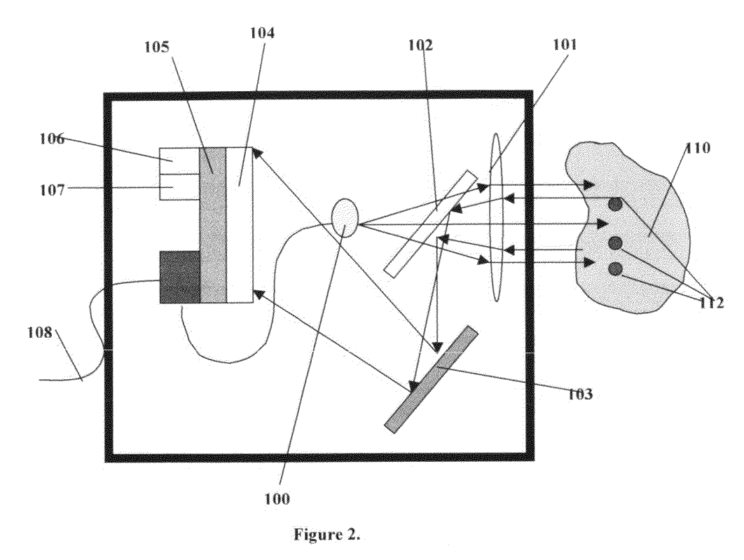

An example of an optical biodosimeter is presented in FIG. 2, wherein the biodosimeter comprises of: an energy source 100, optical lens 101, dichroic filter 102, spectral element 103, CCD photon detector 104 with microprocessor and electronics 105, data communication port 106, on/off power switch 107, power supply 108. The energy source 100, illuminates a body 110 which in response emits light which is reflected by dichroic filter 102 to spectral element 103 and detected by CCD photon detector 104. In this invention it is proposed that energy source 100 is a multispectral source of plurality light emitting diodes and/or laser diodes arranged in a single layer or multiple layers, and each layer is independently controlled. The multiple layer arrangement allows for uniform spectral and spatial distribution and spectral control (U.S. patent application Ser. No. 10/366,267). The biodosimeter interrogates the body 110 with different wavelengths from ultraviolet to infrared, and for each wavelength, the CCD photon detector registers reflectance spectrum of the body 110. Collected spectra are analyzed by software to determine a dose absorbed by the body 110. The dose is accurately calculated by implementing reference data from the body 110. Conditions for collection of reference data can be experimentally defined, and for example, our initial experiments are showing that radiated hair and non-radiated hair excited above 650 nm have the same fluorescence based lines and at lower excitations these hairs display unique spectral signatures. The invention considers the collection of different spectral techniques, such as fluorescence, Raman, Raleigh, but not limited to them.

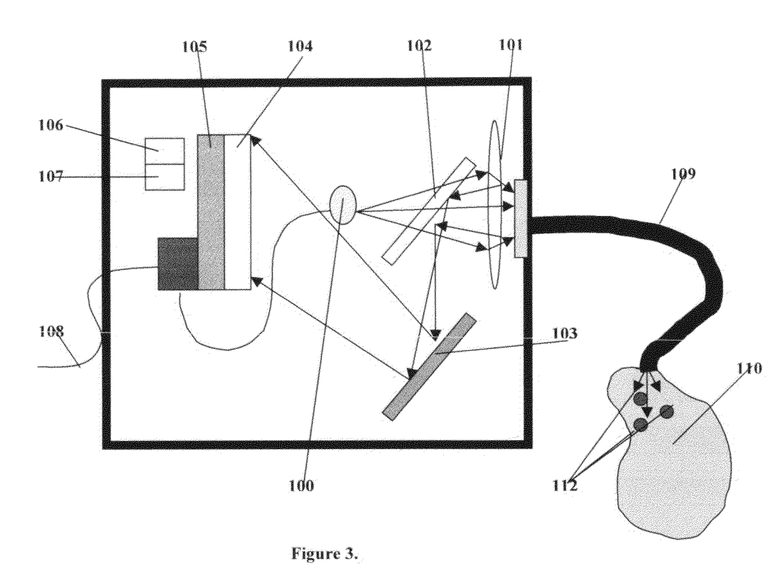

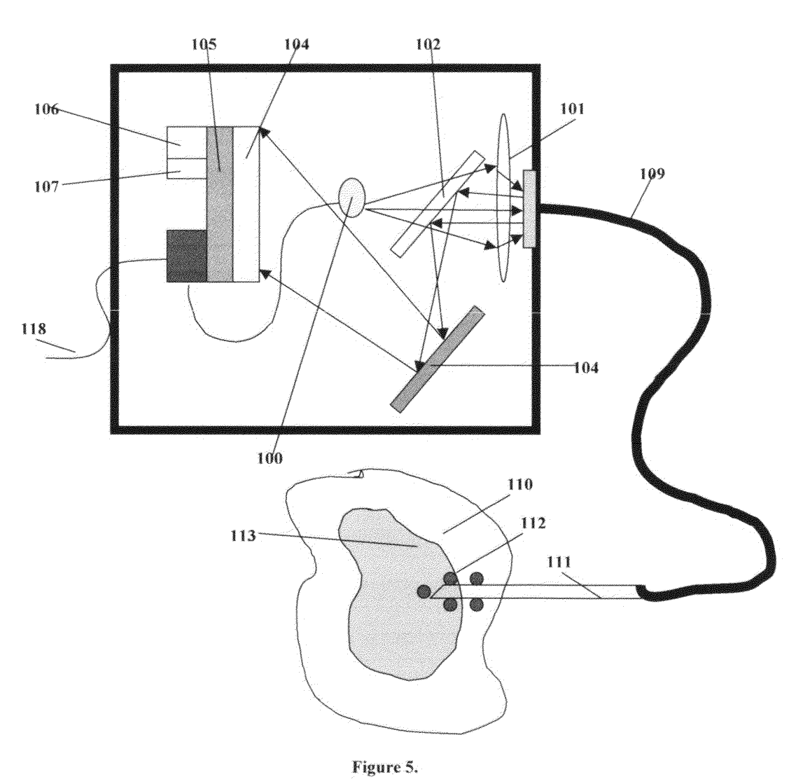

The biodosimeter presented in FIG. 2 may have different designs that depend on applications. As an example, FIG. 3 shows a biodosimeter with a fiber optic 109. Such a design is very useful in field conditions for example, where ambient light may change the measurement accuracy of the radiation dose. The fiber optic biodosimeter is also proposed in the use of dose measurements of the internal body organs. In this case, the fiber optic is replaced with an endoscope 111 (FIG. 5), where the optical end of this endoscope is covered with metal nanoparticles 112 (U.S. patent application Ser. No. 10/916,560) enhancing the spectroscopic signal from the internal organs 113.

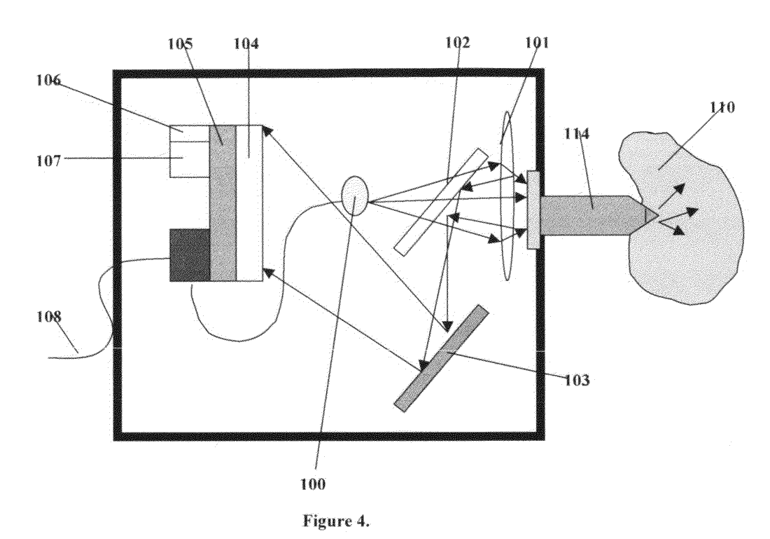

The biodosimeters presented in FIGS. 2 and 3 measure the dose from macro-sized areas, however there is also a need to measure the dose from micro-sized areas. FIG. 4 shows a microscopic objective 110 implemented into the biodosimeter for measurements of micro-objects. For example, the dosimeter equipped with an objective can measure cellular concentrations of oxy-deoxy-hemoglobin and bilirubin to calculate the exposure of the body. Measurements of other body fluids for radiation exposure are anticipated in this invention. The disclosed in this invention of biodosimeters, show almost in real-time, radiation and/or biological-chemical substances exposures of the body. Therefore, the biodosimeter can be integrated with therapy devices or other medical devices for monitoring and recording radiation and/or biological-chemical substances exposure absorbed by the body.

The disclosed biodosimeters are also capable of assessing radiation and/or biological-chemical substances damage to the body and of evaluating healthy conditions of body tissue. These additional functions of biodosimeters can be very helpful in cosmetology, dermatology, and medicine. It is anticipated in this invention to design biodosimeters for such mentioned applications. Essentially, these dosimeters require only different types of software to run the biodosimeters and calculates tissue conditions.

One of the embodiments of this invention includes measurements of the radiation and/or biological-chemical substances doses of the body with techniques other than spectroscopy techniques such as electric, sonic, magnetic, thermal, or electrostatic. The irradiated body experiences changes in biochemical and physical conditions. It is well known that ionizing radiation generates radicals in the body as well as free electrons. Also affected are other metabolic pathways which may change body conductivity, body fluids content, thermal or sonic response, or magnetic properties. Therefore it is well justified to disclose here these techniques as useful techniques for radiation exposure assessment. In addition, the composition of these techniques with spectroscopic techniques also provide useful tools in biodosimetry.

Another embodiment of the invention is related to the use of external substances other than metal nanoparticles to enhance the sensitivity of the proposed biodosimeter. The proposed photosensitizing substance, biorecognitive substance, or fluorescence markers are useful in biodosimetry if they are properly designed. For example, ionized radiation destroys the amino acid Tyrosine into dopamine compounds. Therefore, having a biorecognitive substance labeled with a fluorescence marker targeting dopamine compounds will enable the quantification of the amount of dopamine compounds in the irradiated body and assessing the radiation exposure. Anyone of ordinary skill in the art will appreciate that the biodosimeter can be used for assessment of body damage, biochemical and physical body conditions, or for diagnosis of illnesses and body abnormalities. The biodosimeter with enhanced sensitivity by plasmon and other substances is capable to detect biochemical changes on molecular level with a low false-positive rate and a high positive detection rate that is essential for correct assessments and diagnoses. The invention includes also the use of the biodosimeter for bacterial and viral detection and identification.

What is claimed is:

1. A method of using signatures of human body, animal body or plant for fast response dosimetry comprising steps of: providing a signature of human body, animal body or plant that was exposured to radiation or biochemicals, and providing a detector with a microprocessor and software, the detector is capable of detection the signature and the detector is capable of calculation from the signature an exposure dose absorbed by human body, animal body or plant, detecting by the detector the signature of human body, animal body or plant that was exposured to radiation or biochemicals; and calculating by the detector the exposure dose absorbed by human body, animal body or plant.

2. The method of claim 1, wherein the exposure dose is a radiation dose or is a biochemical dose.

3. The method of claim 1, wherein the signature is a biochemical signature or a physical signature.

4. The method of claim 1, wherein the signature is further plasmon enhanced by a metal nanoparticle, the nanoparticle is in contact with the signature and is excited by a plasmon energy source.

5. The method of claim 1, wherein the detector is detecting the signature by one of the selected technique: spectroscopic, electric, sonic, magnetic, electrostatic or thermal.

6. (canceled)

7. The method of claim 4, wherein the metal nanoparticle is further combined with other substances selected from the group of: a photosensitizing substance, biorecognitive substance, or fluorescence marker.

8. The method of claim 4, wherein the plasmon energy source is a single member energy source or multiple member of energy sources selected from the group of: electromagnetic, electric, electrostatic, magnetic, or thermal.

9. (canceled)

10. The method of claim 8, wherein the electromagnetic plasmon energy source is selected from the group of: light-emitting diode, laser diode, organic light-emitting diode, laser, or lamp.

11. A fast response biodosimeter comprises of: an endoscope with a permanently coated plasmon inducing substance; a plasmon energy source; and a detector with a microprocessor and software, the endoscope is designed to contact/penetrate human body animal body or plant and to be powered by the plasmon energy source to plasmon excite the plasmon inducing substance, the detector is coupled with the endoscope and the detector is capable of detecting a signature and assessing from the signature an exposure dose that was absorbed by human body, animal body or plant.

12. The biodosimeter of claim 11, wherein said plasmon inducing substance is a metal nanoparticle.

13. The biodosimeter of claim 11, wherein said plasmon energy source is a single member energy source or a multiple member energy source selected from the group of: electromagnetic, electric, electrostatic, magnetic, or thermal.

14. The biodosimeter of claim 13, wherein the electromagnetic plasmon energy source or plurality electromagnetic energy sources are is selected from the group of: light-emitting diode, laser diode, organic light-emitting diode, laser, or lamp.

15. The biodosimeter of claim 11, wherein the signature is a biochemical signature or a physical signature.

16. The biodosimeter of claim 11, wherein said plasmon inducing substance is further combined with another substance selected from the group of: a photosensitizing substance, biorecognitive substance, or fluorescence marker.

17. The biodosimeter of claim 11, wherein the detector is capable of detecting the signature by one of the selected technique: spectroscopic, electric, sonic, magnetic, electrostatic or thermal.

18. The biodosimeter of claim 11, wherein the exposure dose is a radiation dose or is a biochemical dose.

19. (canceled)

20. The biodosimeter of claim 11, wherein said biodosimeter is further comprising of a therapy device or a medial device.
